(19) United States
(12) Patent Application Publication
(10) Pub. No.: US 2023/0024058 A1
(43) Pub. Date: Jan. 26, 2023

(54) COMPOSITION THAT PROMOTE PRO-RESOLVING MEDIATORS

(71) Applicant: ACASTI PHARMA INC., Laval (CA)

(72) Inventors: Pierre LEMIEUX, Laval (CA); Jean-François LAPOINTE, Laval (CA); Sarya AZIZ, Laval (CA)

(21) Appl. No.: 17/757,409

(22) PCT Filed: Jan. 8, 2021
(86) PCT No.: PCT/CA2021/050011

§ 371 Date: Jun. 15, 2022

Related U.S. Application Data
(60) Provisional application No. 62/959,196, filed on Jan. 10, 2020.

(51) Int. Cl. A61K 31/683; A61K 31/155; A61K 31/232; A61P 3/10; A61P 1/16

(52) U.S. Cl. CPC A61K 31/683; A61K 31/155; A61K 31/232; A61P 3/10; A61P 1/16

(57) ABSTRACT

It is provided a method for stimulating the secretion of specialized pro-resolving mediators in a subject after the administration of a therapeutic phospholipid compositions such as CaPre®, wherein the composition also increases plasma levels of 17S-HDHA, PDX and 18RS-HEPE in the subject. Increased levels of protectins and resolvins are useful in protecting against many inflammatory-related diseases.

### CROSS-REFERENCE TO RELATED APPLICATION

The present application is claiming priority from U.S. Provisional Application No. 62/959,196 filed Jan. 10, 2020, the content of which is hereby incorporated by reference in its entirety.

### TECHNICAL FIELD

It is provided a method for increasing specialized pro-resolving concentrations (SPM) for the prevention and the treatment of inflammatory-related diseases or conditions by administering a therapeutic phospholipid composition.

### BACKGROUND

Inflammation is a common pathogenesis of many chronic diseases, including cardiovascular and liver diseases, diabetes, arthritis and cancer.

Resolvins and protectins are families of lipid mediators generated from n−3 polyunsaturated fatty acids. E-series resolvins are derived from eicosapentaenoic acid (EPA) and D-series resolvins protectins/neuroprotections and maresins are all derived from docosahexaenoic acid. Studies have shown that SPM increase with time during the inflammatory process, acting on a number of G-coupled protein receptors to affect resolution of inflammation (Barden et al., 2014, Journal of lipid research, 55(11): 2401-2407). Therefore, SPM play an important role in a number of human conditions associated with inflammation.

In view of the severity of these diseases, and the lack of proven and potent treatments, it is thus desirable to develop new treatments for such conditions.

### SUMMARY

It is provided a therapeutic phospholipid composition for increasing specialized pro-resolving concentrations in a subject.

It is further provided a method for increasing specialized pro-resolving concentrations in a subject comprising administering to said subject a therapeutic phospholipid composition.

In an embodiment, the therapeutic phospholipid composition is a concentrated therapeutic phospholipid composition.

In an embodiment, the composition encompassed herein comprises
compounds of the Formula I:

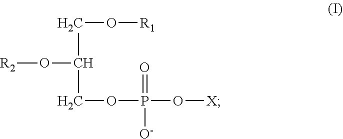

wherein R₁ and R₂ each independently represent a docosahexaenoic acid (DHA) or an eicosapentaenoic acid (EPA) residue; and

wherein each X is independently selected from —CH₂CH₂NH₃, —CH₂CH₂N(CH₃)₃ and

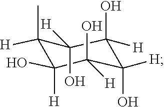

and
free EPA and free DHA.

In an embodiment, the total free and bound EPA in the composition is at a concentration of between 15% and 25% (w/w), and the total free and bound DHA in the composition is at a concentration of between 10% and 15% (w/w).

In another embodiment, the composition is a krill oil composition.

In a further embodiment, the total phospholipids in the composition are at a concentration of at least 50% (w/w (phospholipids/composition)).

the total phospholipids in the composition is at a concentration of at least 55% (w/w (phospholipids/total composition)).

In a further embodiment, the total phospholipids in the composition is at a concentration of at least 60% (w/w (phospholipids/total composition)).

In an additional embodiment, the total phospholipids in the composition is at a concentration of at least about 66% (w/w (phospholipids/total composition)).

In another embodiment, the total phospholipids in the composition is at a concentration of 55-90% (w/w (phospholipids/total composition)).

In a further embodiment, the composition comprises triglycerides in a concentration of below about 5% (w/w).

In an embodiment, the composition further increases plasma levels of 17S-HDHA in said subject.

In another embodiment, the composition further increases plasma levels of PDX in said subject.

In an additional embodiment, the composition further increases plasma levels of 18RS-HEPE in said subject.

In a particular embodiment, the composition is CaPre®.

In a further embodiment, the method described herein further comprises administering about 2 g/day to 4 g/day of therapeutic phospholipid composition to said subject.

In a further embodiment, the method described herein further comprises administering in combination with metformin or Vascepa®.

In a further embodiment, the composition described herein is for further preventing or treating nonalcoholic fatty liver disease (NAFLD) and/or nonalcoholic steatohepatitis (NASH; metabolic steatohepatitis) in said subject.

In an embodiment, the composition described herein is for further increasing insulin secretion from ß-cells in said subject.

In a further embodiment, the composition described herein is for further preventing or treating inflammatory-related diseases or conditions.

### BRIEF DESCRIPTION OF THE DRAWINGS

Reference will now be made to the drawings.

FIG. 1 illustrates the insulin production in (A) in relation with c-peptide levels in (B) wherein Control=HF (high fat) diet placebo treatment, Low Dose=HF fed+NKPL66 (2 g/day), High Dose=HF fed+NKPL66 (4 g/day), Vascepa®=High HF fed+Vascepa®, Metformin=HF fed+Metformin 100 mg/kg/day. Values are means±SEM, n=15 for HF-fed groups and n=6 for Reference.

FIG. 2 illustrates plasma levels of eicosapentaenoic acid (EPA) in (A) and 18RS-HEPE in (B) (a metabolite of EPA and a precursor of Resolvin E1), wherein Low Dose=HF fed+NKPL66 (2 g/day), High Dose=HF fed+NKPL66 (4 g/day), Vascepa®=High HF fed+Vascepa® (4 g/day), Metformin=HF fed+Metformin 100 mg/kg/day. Values are means±SEM, n=15 for HF-fed groups and n=6 for Reference.

FIG. 3 illustrates plasma levels of docosahexaenoic acid (DHA) in (A), 17S-HDHA in (B) and PDX in (C) (two metabolites of DHA), wherein Low Dose=HF fed+NKPL66 (2 g/day), High Dose=HF fed+NKPL66 (4 g/day), Vascepa®=High HF fed+Vascepa® (4 g/day), Metformin=HF fed+Metformin 100 mg/kg/day. Values are means±SEM, n=15 for HF-fed groups and n=6 for Reference.

### DETAILED DESCRIPTION

It is provided a method for increasing specialized pro-resolving concentrations (SPM) for the prevention and the treatment of inflammatory-related diseases or conditions by administering a therapeutic phospholipid composition.

Herein is provided a first evidence of the therapeutic potential of the phospholipid composition in resolving inflammation in insulin resistance, fatty liver disease, a prevalent condition caused by fat accumulation in liver cells and hypertriglyceridemia, a condition characterized by triglycerides levels above or equal to 500 mg/dl.

As encompassed herein, it is encompassed that the therapeutic phospholipid composition is a krill oil composition.

Krill oil compositions have been described as providing beneficial effects in human such as decreasing cholesterol, inhibiting platelet adhesion, inhibiting artery plaque formation, preventing hypertension, controlling arthritis symptoms, enhancing transdermal transport, reducing the symptoms of premenstrual symptoms or controlling blood glucose levels in a patient (WO 02/102394).

U.S. Pat. No. 9,028,877 describes extracts from Antarctic krill having high levels of astaxanthin, phospholipids, including enriched quantities of ether phospholipids, and omega-3 fatty acids. More particularly, U.S. Pat. No. 9,028,877 discloses a method for processing freshly caught krill at the site of capture such as on board of a ship in order to minimize processing of frozen krill that are transported from the capture site to the processing site, which transportation is expensive and may result in the degradation of the krill starting material. The krill is first subjected to a protein denaturation step, such as a heating step, to avoid the formation of enzymatically decomposed oil constituents, such as free fatty acids.

International application no. WO 2011/050474 discloses concentrated therapeutic phospholipid (PL) compositions, comprising for example about 60% w/w phospholipids. These concentrated therapeutic phospholipid compositions are produced using krill oil starting materials as described in U.S. patent application no. 2010/0143571 and U.S. Pat. No. 9,028,877. Such krill oil starting materials do not allow to yield an economically viable commercial amount of these concentrated therapeutic PL compositions for use in the pharmaceutical industry in a consistent manner.

In an embodiment, the therapeutic phospholipid composition is a concentrated phospholipid composition.

More particularly, CaPre® is a krill oil derived composition containing polyunsaturated fatty acids (PUFAs), primarily composed of omega-3 fatty acids, principally eicosapentaenoic acid (EPA) and docosahexaenoic acid (DHA) which is clinical development. In two Phase 2 studies, CaPre achieved a statistically significant reduction of triglycerides and non-HDL cholesterol levels in patients across the dyslipidemia spectrum from patients with mild to moderate hypertriglyceridemia (patients with TG blood levels between 200 mg/dl and 500 mg/dl) to patients with severe hypertriglyceridemia (those with TG levels above or equal 500 mg/dl). Furthermore, in the Phase 2 studies, CaPre demonstrated the potential to actually reduce LDL, or “bad cholesterol”, as well as the potential to increase HDL, or “good cholesterol”.

As provided herewith, it is demonstrated that CaPre (NKPL66) increased insulin production by ß-cells in association with increased c-peptide levels in a dose responsive manner where the higher the dose the more insulin was secreted (see FIG. 1)

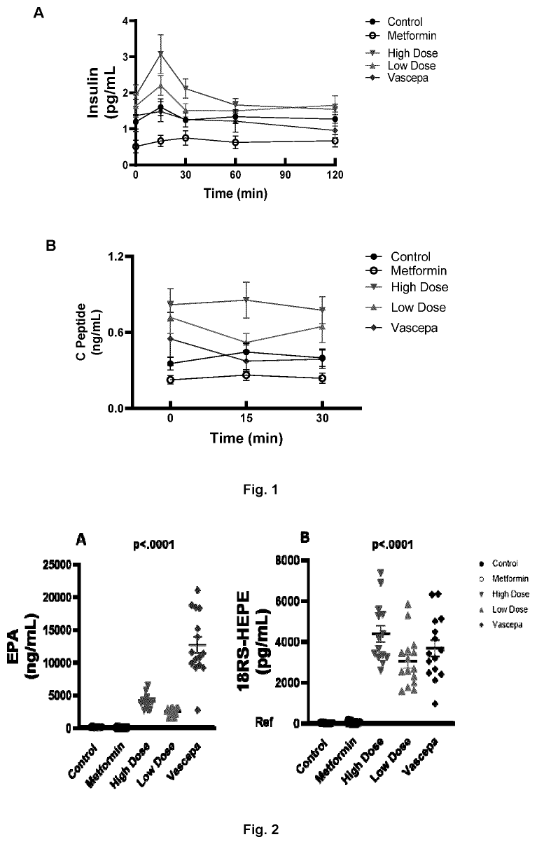

On the other hand, All omega-3 treatments significantly increased plasma 18RS-HEPE (a metabolite of EPA and a precursor of Resolvin E1) versus control. High dose CaPre significantly increased plasma 18RS-HEPE versus Low dose CaPre; see FIG. 2.

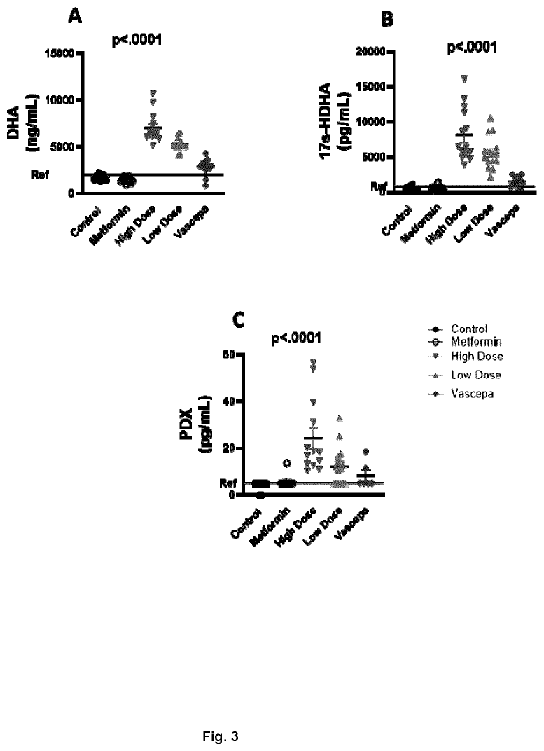

CaPre significantly increased plasma levels of 17S-HDHA and PDX (two metabolites of DHA; see FIG. 3). FIG. 3A shows that all omega-3 treatments significantly increased plasma DHA vs Control and Metformin. High Dose CaPre significantly increased plasma DHA versus Vascepa® and Low Dose CaPre. Low Dose CaPre significantly increased DHA versus Vascepa®. FIG. 3B shows that both High and Low Dose CaPre significantly increased plasma 17S-HDHA versus Control, Metformin and Vascepa®. High dose CaPre significantly increased plasma 17S-HDHA versus Low Dose CaPre. FIG. 3C shows that both High and Low Dose CaPre significantly increased plasma PDX versus Control. High Dose CaPre significantly increased plasma PDX versus Low Dose CaPre, Vascepa® and Metformin.

Resolvins belong to a class of polyunsaturated fatty acid (PUFA) metabolites termed specialized proresolving mediators (SPMs). Resolvins are metabolic byproducts of omega-3 fatty acids, primarily EPA and DHA, as well as docosapentaenoic acid (DPA) and clupanodonic acid. Resolvins are believed to be involved in promoting restoration of normal cellular function following the inflammation that occurs after tissue injury.

Protectin DX (PDX) is an isomer of protectin/neuroprotectin DI, which is derived from omega-3 fatty acid DHA (docosahexaenoic acid) having anti-inflammatory and anti-diabetic properties.

As demonstrated herein, an increased insulin production in association with increased c-peptide levels is observed with increasing doses of CaPre demonstrating that this effect is linked to continuous elevated insulin secretion by ß-cells. CaPre exhibited a dose response where the higher the dose the more insulin was secreted.

Both CaPre and icosapent ethyl significantly increased plasma 18RS-HEPE, as compared to the untreated control and metformin groups. Despite the lower levels of EPA in CaPre's composition, the actual levels of 18RS-HEPE reached in the blood were higher for CaPre than levels produced by icosapent ethyl. Again, a dose response effect was seen with CaPre. 18RS-HEPE and Resolvin E1 are both resolving mediators of OM3s, and particularly EPA, and they are involved in the resolution of inflammation that is triggered in many chronic diseases including obesity and diabetes.

As exemplified herein, both high dose (HED or human equivalent dose of 4 grams/day), and low dose (HED of 2 g/day) of CaPre significantly increased plasma levels of 17S-HDHA and PDX as compared to the untreated control group. The effects of high dose CaPre on PDX was very robust and significant, and much greater than those of icosapent ethyl, which showed virtually no response. Research has shown that increased levels of PDX improves insulin sensitivity in various models of insulin resistance and diabetes by several mechanisms, including by limiting inflammation in metabolic tissues, as well as by enhancing skeletal muscle IL-6 secretion, AMP activated protein kinase (AMPK) activation and glucose uptake, and by enhancing insulin's ability to suppress hepatic glucose production, which is also elevated in diabetic patients.

It has been described in U.S. 2019/0070140 the effects of PDX upon lipid metabolism and TG accumulation in a hyperlipidemia status. It is known that PDX mediate a protective mechanism against palmitate-induced ER stress and fatty liver in HepG2 liver cells. Further, according to experiments on animal models to study influence of PDX upon ER stress, fatty liver and hyperlipidemia, it is disclosed in U.S. 2019/0070140 that PDX is a drug capable of being used as a therapeutic agent for hyperlipidemia and ER stress-mediated diseases.

In an embodiment, said therapeutic phospholipid composition comprises
compounds of the Formula I:

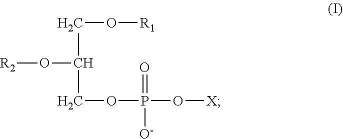

wherein R₁ and R₂ each independently represent a docosahexaenoic acid (DHA) or an eicosapentaenoic acid (EPA) residue; and

wherein each X is independently selected from —CH₂CH₂NH₃, —CH₂CH₂N(CH₃)₃ and

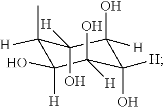

and
free EPA and free DHA,

wherein the total phospholipids in the composition are at a concentration of at least 50% (w/w (phospholipids/composition)).

In a further embodiment, the total free and bound EPA in the composition is at a concentration of between 15% and 25% (w/w), and the total free and bound DHA in the composition is at a concentration of between 10% and 15% (w/w).

In another embodiment, the total phospholipids in the composition are at a concentration of 55% (w/w (phospholipids/total composition)).

In an embodiment, the total phospholipids in the composition are at a concentration of 60% (w/w (phospholipids/total composition)).

In an embodiment, the total phospholipids in the composition are at a concentration of 66% (w/w (phospholipids/total composition)).

In another embodiment, the total phospholipids in the composition are at a concentration of 90% (w/w (phospholipids/total composition)).

In an additional embodiment, the composition comprises triglycerides in a concentration of below about 5% (w/w).

The term “about” when used in this disclosure along with a recited value means the value recited and includes the range of + or −5% of the value. For example, the phrase about 60% means 60% and + or −5% of 60, i.e. 56% to 64%.

In an embodiment, the composition is CaPre®.

In a further embodiment, the composition further increases plasma levels of 17S-HDHA, of PDX, and/or 18RS-HEPE in the subject.

It is encompassed that the composition encompassed herein further prevents or treats nonalcoholic fatty liver disease (NAFLD) and/or nonalcoholic steatohepatitis (NASH; metabolic steatohepatitis) in the subject.

Administration of the therapeutic phospholipid compositions as encompassed herein can be accomplished via any mode of administration for therapeutic agents. These modes include systemic or local administration such as oral, parenteral, transdermal, subcutaneous, or topical administration modes. Preferably, the phospholipid composition is administered orally.

Depending on the intended mode of administration, the compositions can be in solid, semi-solid or liquid dosage form, such as, for example, injectables, tablets, pills, time-release capsules, elixirs, tinctures, emulsions, syrups, liquids, suspensions, or the like, sometimes in unit dosages and consistent with conventional pharmaceutical practices. Likewise, they can also be administered in intravenous (both bolus and infusion), intraperitoneal, subcutaneous or intramuscular form, all using forms well known to those skilled in the pharmaceutical arts.

Illustrative pharmaceutical compositions are tablets and gelatin capsules comprising a therapeutic phospholipid composition neat, or if required, contains a pharmaceutically acceptable carrier, such as a) a diluent, e.g., purified water, triglyceride oils, such as hydrogenated or partially hydrogenated vegetable oil, or mixtures thereof, corn oil, olive oil, sunflower oil, safflower oil, lactose, dextrose, sucrose, mannitol, sorbitol, cellulose, sodium, saccharin, glucose and/or glycine; b) a lubricant, e.g., silica, talcum, stearic acid, its magnesium or calcium salt, sodium oleate, sodium stearate, magnesium stearate, sodium benzoate, sodium acetate, sodium chloride and/or polyethylene glycol; for tablets also; c) a binder, e.g., magnesium aluminum silicate, starch paste, gelatin, tragacanth, methylcellulose, sodium carboxymethylcellulose, magnesium carbonate, natural sugars such as glucose or beta-lactose, corn sweeteners, natural and synthetic gums such as acacia, tragacanth or sodium alginate, waxes and/or polyvinylpyrrolidone, if desired; d) a disintegrant, e.g., starches, agar, methyl cellulose, bentonite, xanthan gum, algiic acid or its sodium salt, or effervescent mixtures; e) absorbent, colorant, flavorant and sweetener; f) an emulsifier or dispersing agent, such as Tween® 80, Labrasol, HPMC, DOSS, caproyl 909, labrafac, labrafil, peceol, transcutol, capmul MCM, capmul PG-12, captex 355, gelucire, vitamin E TGPS or other acceptable emulsifier; and/or g) an agent that enhances absorption of the compound such as cyclodextrin, hydroxypropyl-cyclodextrin, PEG™400, or PEG™200.

The dosage regimen utilizing the therapeutic phospholipid compositions is selected in accordance with a variety of factors including type, species, age, weight, sex and medical condition of the subject; the severity of the condition to be treated; the route of administration; the renal or hepatic function of the subject; and the particular therapeutic phospholipid composition employed. A physician or veterinarian of ordinary skill in the art can readily determine and prescribe the effective amount of the drug required to prevent, counter or arrest the progress of the condition.

The therapeutic phospholipid compositions can be administered in a single daily dose, or the total daily dosage can be administered in divided doses of two, three or four times daily. In some embodiments, of combination therapy, the therapeutic phospholipid composition and the therapeutic agent can be administered simultaneously. In other embodiments, the therapeutic phospholipid composition and the therapeutic agent can be administered sequentially. In still other embodiments of combination therapy, the therapeutic phospholipid composition can be administered daily and the therapeutic agent can be administered less than daily. In still other embodiments of combination therapy, the therapeutic phospholipid composition can be administered daily and the therapeutic agent can be administered more than once daily.

Preferably, about 2 g/day to 4 g/day are administered.

In an embodiment, the composition encompassed herein is administered in combination with metformin or Vascepa®.

### Example I

### Effect of CaPre on Glucose Tolerance and Insulin Sensitivity in Prediabetes Mouse Model

The purpose of this study was to induce obesity and Type 2 Diabetes Metillus in mice and test their response after treatment with CaPre in comparison to a Standard of Care (SOC) diabetes treatment (Metformin) and to an EPA only ethyl esters treatment for hypertriglyceridemia (Vascepa®). C57BL/6J mice were fed with high-fat high-sugar (HFHS) diet for 2 weeks, followed by 12 weeks of treatment with CaPre administered orally (2 g or 4 g HED) compared to Metformin, Vascepa® or vehicule. Mice were on a HFHS diet throughout the 12 weeks, their bodyweight was monitored every 2 days, and food intake measured every 3 days. As a control, an additional treatment group will be on chow diet throughout the entire study. After 12 weeks of CaPre, mice will be sacrificed and analyzed for:
Insulin injection (insulin signaling measurement)
Fasting plasma collection (Cardiac puncture)
Tissue collection: Adipose tissue depots (subcutaneous, visceral and brown), 3 muscles, liver, pancreas, intestine and caecum contents, heart and aorta. Each tissue will be divided into: one section for mRNA (RNA selector), one section for histological analysis (paraformaldehyde) and one section flash frozen for lipidomics/inflammatory markers/protein analysis

The proposed analyses conducted are:
Plasma measurements of Total cholesterol (TC), TG, high-density lipoprotein cholesterol (HDL-c), low-density lipoprotein cholesterol (LDL-c), alanine aminotransferase (ALT), aspartate aminotransferase (AST) (pre/post treatment).
Determine insulin and c-peptide profile during oral glucose tolerance test (OGTT).
Insulin signaling by immunoblot assay in liver, visceral adipose tissue and muscle.
Diabetes biomarkers in the plasma by multiplex analysis.
Lipidomics (plasma, liver), EPA, DHA, DPA, resolvins and protectins.
Microbiota analysis.

### Example II

### Effect of CaPre on Hyperlipidemia and Hepatic Metabolism in a Prediabetes/Type 2 Diabetes Metillus Setting

The purpose of this study will be the use of the LDLr−/−ApoB100 mouse model which combines insulin resistance and LDL cholesterol-driven hyperlipidemia. 6-week old LDLr−/−ApoB100 mice will be fed with HFHS diet for 2 weeks, followed by 12 weeks of treatment with CaPre administered orally (2 g or 4 g HED) compared to Metformin, Vascepa® or vehicule. The mice will be on HFHS diet throughout the 12 weeks, their bodyweight will be monitored every 2 days and food intake will be measured every 3 days. As a control, an additional treatment group will be on chow diet throughout the entire study.

After 12 weeks of CaPre treatment, mice will be sacrificed and analyzed for:
Fasting plasma collection (cardiac puncture)
Tissue collection: Adipose tissue depots (subcutaneous, visceral and brown) 3 muscles, liver, pancreas, intestine and caecum contents, heart and aorta. Each tissue will be divided into: 1 section for mRNA (RNA selector), 1 section for histological analysis (paraformaldehyde) and 1 section flash frozen for lipidomics/inflammatory markers/protein analysis.

The proposed analyses conducted will be:
Plasma measurements of Total cholesterol (TC), TG, high-density lipoprotein cholesterol (HDL-c), low-density lipoprotein cholesterol (LDL-c), alanine aminotransferase (ALT), aspartate aminotransferase (AST) (pre/8 week/post treatment);
Plasma measurement of apolipoprotein B (ApoB) and lipoprotein-associated phospholipase A2 (Lp-LpA2);
Gene expression of TG regulation, de novo lipogenesis, lipoprotein metabolism and bile acid synthesis;
Inflammatory markers by multiplex analysis (liver and plasma);
Histology in liver for Non-alcoholic fatty liver disease (fibrosis, steatosis)
Liver lipids (Triglycerides (TG), Total cholesterol (TC), cholesterol esters (CE), free cholesterol (FC) and phosphatidylcholine (PC));
Bile acids; and
Microbiota analysis.

This study provides demonstration of CaPre efficacy on plasma lipid profile and in fatty liver disease by further comparing the impact of CaPre on plasma TGs, LDL-C and HDL-C as well as on hepatic lipid accumulation versus that of isocapent ethyl (Vascepa®) and metformin.

### Example III

### Phase III Study of CaPre in Patients with Severe Hypertriglyceridemia in Multi-Center, Placebo-Controlled, Randomized, Double-Blind Trial

The purpose of this study was to compare the efficacy and safety of CaPre 4 g daily with placebo in lowering fasting TG levels and other lipid parameters in patients with severe HTG (fasting TG levels 500 mg/dl and 1,500 mg/dl). The primary endpoint was the percent change from baseline in fasting TG levels after 12 weeks of treatment. Key secondary endpoints included percent change from baseline in non-HDL-C, VLDL-C (ultracentrifugation), HDL-C and LDL-C (ultracentrifugation) after 12 weeks of treatment. Pro-resolving mediators (i.e. resolvins, protectin DX) will be measured.

While the present disclosure has been described in connection with specific embodiments thereof, it will be understood that it is capable of further modifications and this application is intended to cover any variations, uses, or adaptations including such departures from the present disclosure as come within known or customary practice within the art and as may be applied to the essential features hereinbefore set forth, and as follows in the scope of the appended claims.

1-18. (canceled)

19. A method for increasing specialized pro-resolving concentrations in a subject comprising administering to said subject a therapeutic phospholipid composition.

20. The method of claim 19, wherein said therapeutic phospholipid composition is a concentrated therapeutic phospholipid composition.

21. The method of claim 19, said composition comprising:
compounds of the Formula I: 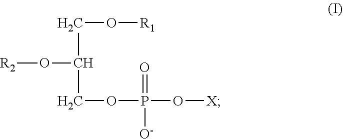
wherein R₁ and R₂ each independently represent a docosahexaenoic acid (DHA) or an eicosapentaenoic acid (EPA) residue; and
wherein each X is independently selected from —CH₂CH₂NH₃, —CH₂CH₂N(CH₃)₃ and 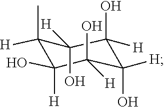
and
free EPA and free DHA.

22. The method claim 19, wherein the total free and bound EPA in the composition is at a concentration of between 15% and 25% (w/w), and the total free and bound DHA in the composition is at a concentration of between 10% and 15% (w/w).

23. The method of claim 19, wherein the composition is a krill oil composition.

24. The method of claim 19, wherein the total phospholipids in the composition are at a concentration of at least 50% (w/w (phospholipids/composition)).

25. The method of claim 19, wherein the total phospholipids in the composition is at a concentration of at least 55% (w/w (phospholipids/total composition)).

26. The method of claim 19, wherein the total phospholipids in the composition is at a concentration of at least 60% (w/w (phospholipids/total composition)).

27. The method of claim 19, wherein the total phospholipids in the composition is at a concentration of at least about 66% (w/w (phospholipids/total composition)).

28. The method of claim 19, wherein the total phospholipids in the composition is at a concentration of 55-90% (w/w (phospholipids/total composition)).

29. The method of claim 19, wherein the composition comprises triglycerides in a concentration of below about 5% (w/w).

30. The method of claim 19, wherein the composition further increases plasma levels of 17S-HDHA in said subject.

31. The method of claim 19, wherein the composition further increases plasma levels of PDX in said subject.

32. The method of claim 19, wherein the composition further increases plasma levels of 18RS-HEPE in said subject.

33. The method of claim 19, wherein the composition is CaPre®.

34. The method of claim 19, comprising administering about 2 g/day to 4 g/day of therapeutic phospholipid composition to said subject.

35. The method of claim 19, further comprising administering in combination with metformin or Vascepa®.

36. The method of claim 19, further preventing or treating nonalcoholic fatty liver disease (NAFLD) and/or nonalcoholic steatohepatitis (NASH; metabolic steatohepatitis) in said subject.

37. The method of claim 19, further increasing insulin secretion from R-cells in said subject.

38. The method of claim 19, further preventing or treating inflammatory-related diseases or conditions.
